(19) United States
(12) Patent Application Publication
(10) Pub. No.: US 2023/0139964 A1
(43) Pub. Date: May 4, 2023

(54) GENOME DASHBOARD

(71) Applicant: The Research Institute at Nationwide Childern's Hospital, Columbus, OH (US)

(72) Inventors: Simon Lin Linwood, Dublin, OH (US); Yungui Huang, Upper Arlington, OH (US); Seyed Scheil Moosavinasab, Columbus, OH (US); Matthew Beiley, Worthington, OH (US); Robert VanDevender Strouse, Columbus, OH (US); Rajeswari Swaminathan, Columbus, OH (US); Katherine Miller, Grove City, OH (US); Jeremy Patterson, Columbus, OH (US); Syed-Amad Hussain, Columbus, OH (US); En Julin, Dublin, OH (US)

(21) Appl. No.: 17/909,539

(22) PCT Filed: Mar. 8, 2021
(86) PCT No.: PCT/US21/21341

§ 371 Date: Sep. 6, 2022

Related U.S. Application Data
(60) Provisional application No. 62/986,164, filed on Mar. 6, 2020.

(51) Int. Cl. G16B 45/00; G16B 20/20

(52) U.S. Cl. CPC G16B 45/00; G16B 20/20; G16H 50/20

(57) ABSTRACT

A genome system for displaying an interactive genome dashboard is provided herein. The genome system includes processing device having a processor configured to perform machine learning and performing a matching function between phenotypes and gene variants to create gene matches based upon multiple text inputs and genome sequences introduced through the interactive genome dashboard. The processing device includes memory wherein previously generated matches are tagged and stored based upon the multiple text inputs, the genome sequence, and subsequent receipt of user interaction with the generated matches.

### CROSS REFERENCES TO RELATED APPLICATIONS

The following application claims priority under 35 U.S.C. § 119 (e) to U.S. Provisional Patent Application Ser. No. 62/986,164 filed Mar. 6, 2020 entitled GENOME DASHBOARD. The above-identified application is incorporated herein by reference in its entirety for all purposes.

### TECHNICAL FIELD

The present disclosure generally relates to genotype to phenotype association methods and devices, and more particularly to genotype to phenotype association methods and devices for use in indexing whole exomes or genomes relative to phenotypic expression.

### BACKGROUND

Clinical genetics is a relatively new and evolving practice. Whole exome sequencing (WES) is increasingly being utilized to establish the genetic basis of disease in patients. Advances in genome sequencing technologies have allowed for rapid development of pipelines for sequence reading, alignment, and variant calling, but the downstream tasks of variant interpretation and assessing the clinical relevance of variants are still being refined.

Clinical genetics are a relatively new and evolving practice. Whole exome sequencing (WES) is increasingly being used in clinical settings to establish the genetic basis of rare and single gene disorders in patients. Sequencing laboratories will return a static report of genetic variants that are potentially associated with a patient's clinical feature (phenotype). The static report does not allow a clinician to easily assess the raw data, update results in light of an appearance of new symptoms. Further, there is an inherent lag between the static report and newly discovered genotype/phonotype associations.

Sometimes referred to as tertiary analysis, variant interpretation includes annotating, filtering, and associating sequence variants with disease, for example, translating the gnomic data into a clinical diagnosis. Typically, WES generates 30-60 million base pairs, or 4-6 GB of raw sequencing data for each patient. After aligning to the human reference genome about 250,000-400,000 variants are identified. Most of the variants are likely to be benign, and only a small number-often as few as one or two-contribute to a specific genetic disease in a patient. Identifying which sequence variants are disease-causing can be overwhelming and difficult for researchers to easily accomplish. Typically, extensive bioinformatics experience is required to use many of the analysis tools currently available to the research community.

Currently, there is a bottleneck in human genomics and exome sequencing studies when narrowing down a list of sequencing variants from 100,000+ to just a few (usually 1 or 2) that are disease-causing in an individual.

### SUMMARY

One aspect of the present disclosure comprises genome system for displaying an interactive genome dashboard. The genome system includes a processing device having a processor configured to perform machine learning and performing a matching function between phenotype keywords and gene variants identified in a genome sequence to create gene matches based upon multiple text inputs and the genome sequence introduced through the interactive genome dashboard. The processing device includes memory wherein previously generated matches are tagged and stored based upon the multiple text inputs, the genome sequence, and subsequent receipt of user interaction with the generated matches. The processing device receives one or more phenotype keywords and the genome sequence from the genome dashboard, identifies genetic variants associated with the phenotype keywords, matches the genetic variants to known genetic variants to generate a first diagnosis, and sends a signal to present the first diagnosis and the phenotype keywords associated with the genetic variants on the genome dashboard. Responsive to receiving a signal adding filters from a user of the genome dashboard, the processing device applies added filters to the phenotype keywords associated with the genetic variants and the first diagnosis and generates filtered phenotype keywords associated with the genetic variants and generates a second diagnosis, and sends a signal to present the second diagnosis and the filtered phenotype keywords associated with the genetic variants on the genome dashboard.

Another aspect of the present disclosure comprises a non-transitory computer readable medium storing instructions executable by an associated processor to perform a method for implementing a genome system for displaying an interactive genome dashboard. The method includes storing a first diagnosis generated by the genome system based upon a genome sequence and initial data, the initial data comprising identified genetic variants of the genome sequence, phenotype keywords, multiple text inputs, and phonotype genetic variant associations. The method further includes, responsive to receiving additional multiple text inputs, extracting one or more additional phonotypic terms from the additional multiple text inputs, identifying one or more genetic variants present in the genome sequence associated with the one or more additional phonotypic terms, and generating a second diagnosis based upon the one or more additional phonotypic terms and the initial data. The method additionally includes responsive to the first diagnosis being the same as the second diagnosis, storing the second diagnosis; and responsive to the first diagnosis being different than the second diagnosis, presenting the second diagnosis on the genome dashboard.

Yet another aspect of the present disclosure comprises A genome system for displaying an interactive genome dashboard. The genome system includes a processing device having a processor configured to perform a matching function between phenotypes and gene variants to create gene matches based upon multiple text inputs and genome sequences introduced through the interactive genome dashboard. The processing device receives one or more phenotype keywords and a genome sequence of a patient exhibiting the one or more phenotype keywords and matches and presents on the interactive genome dashboard one or more gene variants present in the genome sequence associated with the one or more phenotype keywords. Further, the processing device identifies and presents on the interactive genome dashboard disease candidates based upon the one or more gene variants association with the one or more phenotype keywords, identifies and presents on the interactive genome dashboard non-represented gene variants that are associated with each of the disease candidates that are not present in the one or more gene variants, and generating sortable list on the interactive genome dashboard of identifying each of the one or more phenotype keywords and each of the one or more gene variants the comprises clinical evidence supporting each of the disease candidates.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals, unless otherwise described refer to like parts throughout the drawings and in which:

FIG. 1A is a schematic diagram of a genome system for supporting a genome dashboard, in accordance with one example embodiment of the present disclosure;

FIG. 1B is a schematic diagram of a method of using a genome dashboard supported by a genome system, in accordance with one example embodiment of the present disclosure;

FIG. 2A illustrates a schematic view of a first view of a genome dashboard, according to one example embodiment of the present disclosure;

FIG. 2B illustrates a first view of a genome dashboard, according to one example embodiment of the present disclosure;

FIG. 3A illustrates a second view of a genome dashboard, according to one example embodiment of the present disclosure;

FIG. 3B illustrates a second view of a genome dashboard, according to one example embodiment of the present disclosure;

FIG. 4A illustrates a schematic view of a third view of a genome dashboard, according to one example embodiment of the present disclosure;

FIG. 4B illustrates a third view of a genome dashboard, according to one example embodiment of the present disclosure;

FIG. 5 is a schematic diagram of a method of using a genome dashboard, according to another example embodiment of the present disclosure;

FIG. 5A is a view of an applied filter illustration in a genome dashboard, according to another example embodiment of the present disclosure;

FIG. 5B is a view of ranked findings in a genome dashboard, according to another example embodiment of the present disclosure;

FIG. 6 is a schematic diagram of a method of using a genome dashboard, including inputs and outputs utilized in presenting ranked and highlighted findings to a user, in accordance with one example embodiment of the present disclosure;

FIG. 7 is a schematic diagram of a method of using a genome dashboard, including incorporating records of user interaction in generating ranked and highlighted findings to present to a user, in accordance with one example embodiment of the present disclosure;

FIG. 8 is a schematic diagram of a method of using a genome dashboard, including generating ranked and illustrated findings from multiple inputs, including plain text inputs, to present a user, in accordance with one example embodiment of the present disclosure;

FIG. 9 is a schematic diagram of a method of using a genome dashboard including incorporating records of user interaction, according to another example embodiment of the present disclosure;

FIG. 10a is a schematic diagram of a method of using a genome dashboard including generating a comparison mode display, according to another example embodiment of the present disclosure;

FIG. 10b is a schematic diagram of a method of using a genome dashboard including generating a best match list, a worst match list, a genes present by disease candidate association list, and/or a most selected diagnosis list, according to another example embodiment of the present disclosure;

FIG. 10c is a schematic diagram of a method of using a genome dashboard including generating views based upon received user input, according to another example embodiment of the present disclosure;

FIG. 10d is an example filter for use with the genome dashboard, according to another example embodiment of the present disclosure;

FIG. 10e is an example of best to worst ranked list for use with the genome dashboard, according to another example embodiment of the present disclosure;

FIG. 11 is a schematic diagram of a method of using a genome dashboard including generating one or more versions of a case for comparison, according to another example embodiment of the present disclosure;

FIG. 12 is a schematic diagram of a method of using a genome dashboard including resetting a case history, according to another example embodiment of the present disclosure; and

FIG. 13 is a schematic diagram of a method of using a genome dashboard including diagnosing multiple genetic conditions, according to another example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure generally relates to genotype to phenotype association methods and devices, and more particularly to genotype to phenotype association methods and devices for use in in indexing whole exomes or genomes relative to phenotypic expression.

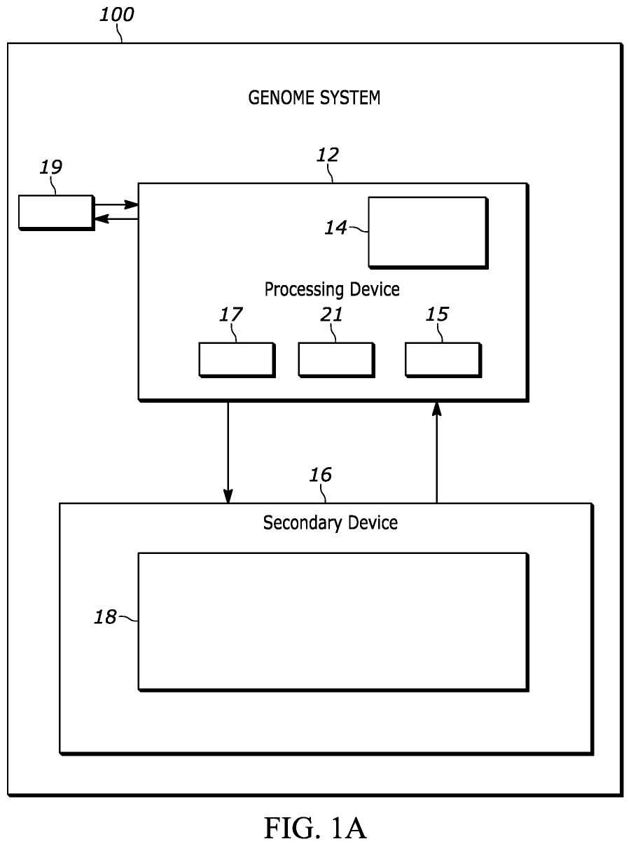

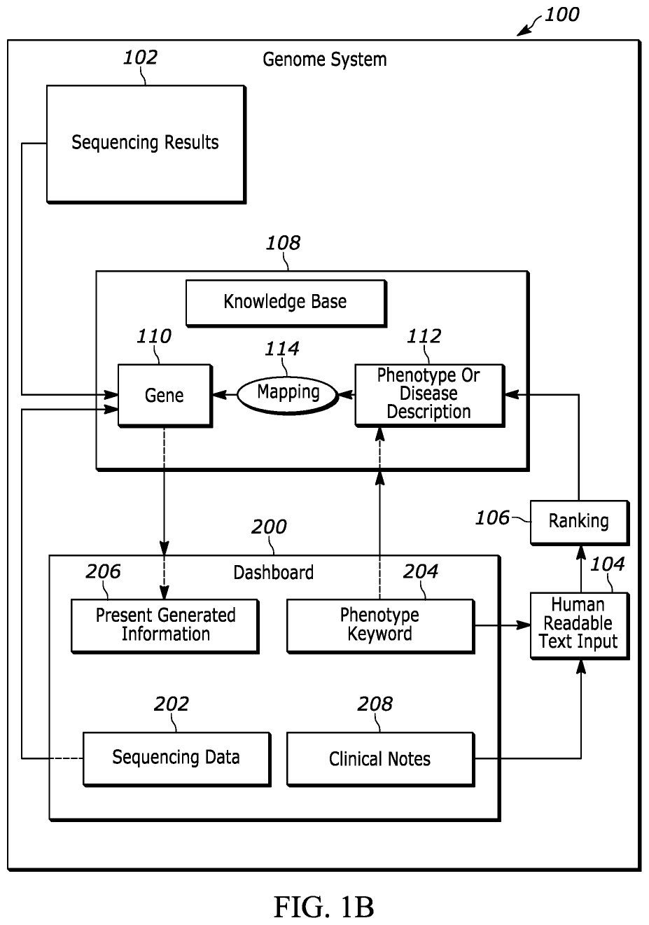

FIG. 1 illustrates a schematic diagram of a genome system 100, in accordance with one of the exemplary embodiments of the disclosure. The genome system 100 includes a processing device 12, which includes a computing device (e.g. a database server, a file server, an application server, a computer, or the like) with computing capability and/or a processor 14. The processor 14 comprises central processing units (CPU), such as a programmable general purpose or special purpose microprocessor, and/or other similar device or a combination thereof.

The processing device 12 would generate outputs based upon inputs received from a secondary device 16, cloud storage, a local input form a user, etc. It would be appreciated by having ordinary skill in the art that the processing device 12 would include a data storage device 17 in various forms of non-transitory, volatile, and non-volatile memories which would store buffered or permanent data as well as compiled programming codes used to execute functions of the processing device 12. In another example embodiment, the data storage device 17 can be external to and accessible by the processing device 12, the data storage device 17 may comprise an external hard drive, cloud storage, and/or other external recording devices 19.

In one example embodiment, the processing device 12 comprises one of a remote or local computer system 21. The computer system includes desktop, laptop, tablet hand-held personal computing device, IAN, WAN, WWW, and the like, running on any number of known operating systems and are accessible for communication with remote data storage, such as a cloud, host operating computer, via a world-wide-web or Internet.

In another example embodiment, the processing device 12 comprises a processor, a data storage, computer system memory that includes random-access-memory (“RAM”), read-only-memory (“ROM”) and/or an input/output interface. The processing device 110 executes instructions by non-transitory computer readable medium either internal or external through the processor that communicates to the processor via input interface and/or electrical communications, such as from the secondary device 16 (e.g., smart phone, tablet, personal computer, or other device). In yet another example embodiment, the processing device 12 communicates with the Internet, a network such as a LAN, WAN, and/or a cloud, input/output devices such as flash drives, remote devices such as a smart phone or tablet, and displays. The secondary device 16 includes a display 18, the display having visual, audio, etc. output. In one example embodiment, the genome system 100 is a web-based tool (e.g., no download or installation is needed to utilize the genome system 100). In another example embodiment, the genome system 100 is partially and/or completely downloadable. The genome system 100 is interactive, meaning a user may change and alter their search preferences and view results in real-time.

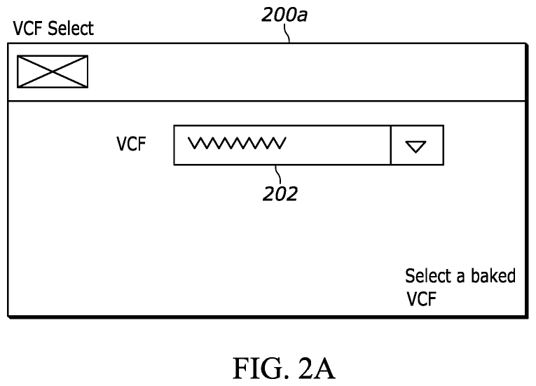

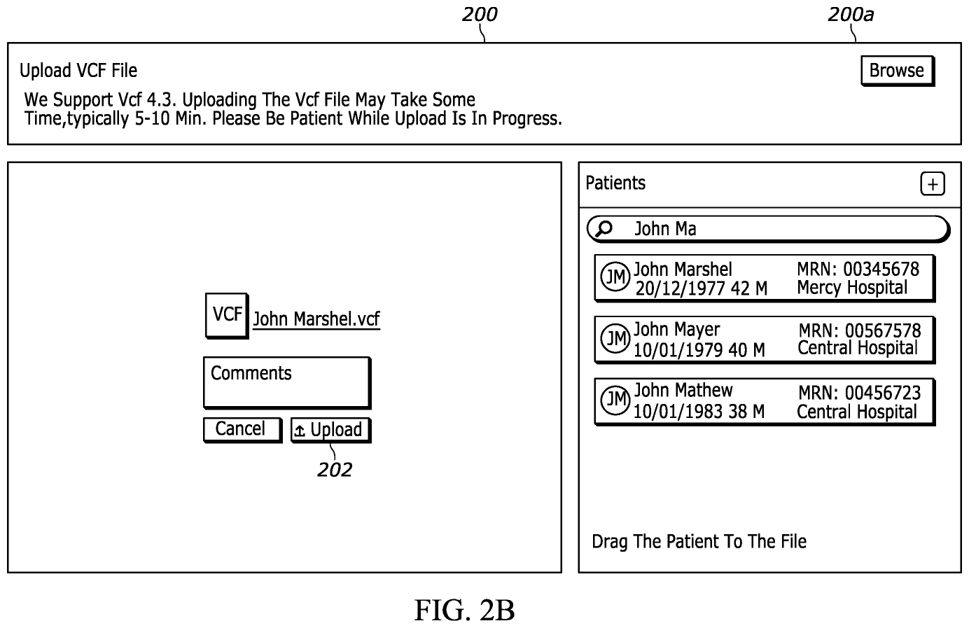

Illustrated in FIGS. 1B, 2A, and 2B, the genome system 100 ingests sequencing data 202 (e.g., variant call format (VCF) files), provided by a user and/or sequencing results 102, provided by a second party gene sequencing unit, so that individual gene variants are analyzed. Genome sequencing data/results 102, 202 are provided to the processing device 12 having a knowledge base, artificial intelligence, and/or machine learning capability. The genome system 100 includes a graphical user interface that comprises a genome dashboard 200 (e.g., displayed on the display 18) that utilizes plain text language to perform searches. At least one of phenotype keyword 204 (e.g., cough, fever, etc.), or clinical notes 208 (e.g., the patient exhibits jaundice, etc.) is input into the genome dashboard 200. In one example embodiment, the phenotype keyword 204 and/or clinical notes 208 are human readable text, wherein a human readable text input 104 is ranked 106 to remove unimportant or clinically irrelevant terms and elevate important clinical terms. The ranked terms 106 are assigned to a phenotype or disease description 112. Genes 110 of a patient are mapped using the phenotype or disease description 112, wherein gene variants or single nucleotide polymorphisms (SNPs) associated with the phenotype or disease description are identified and a grouping of the gene variants with the phenotype or disease description 112 are generated. The grouping is presented as generated information 206 on the genome dashboard.

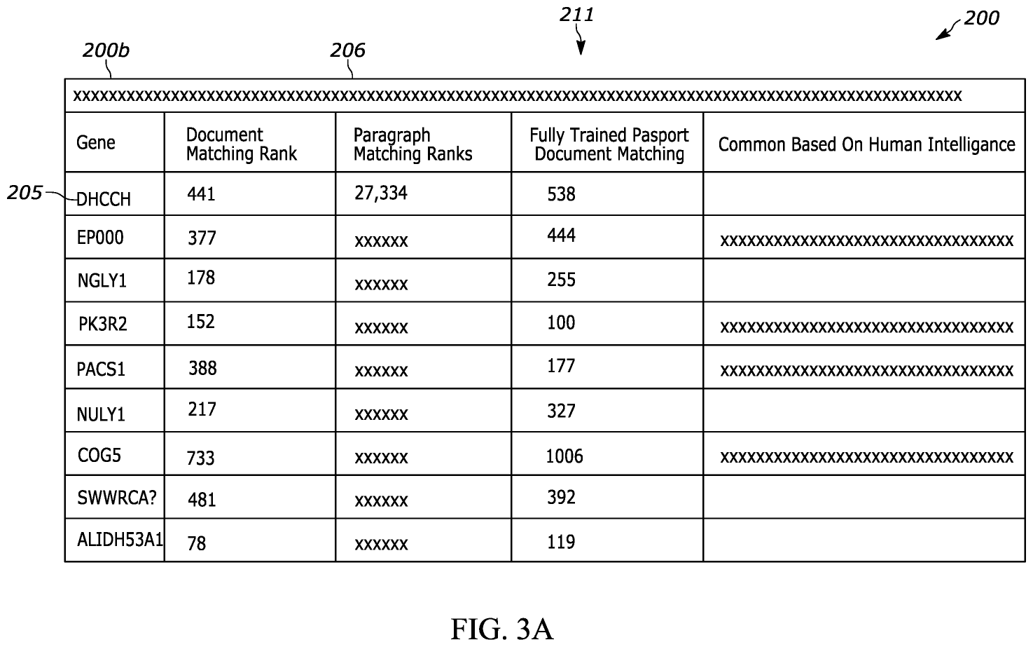

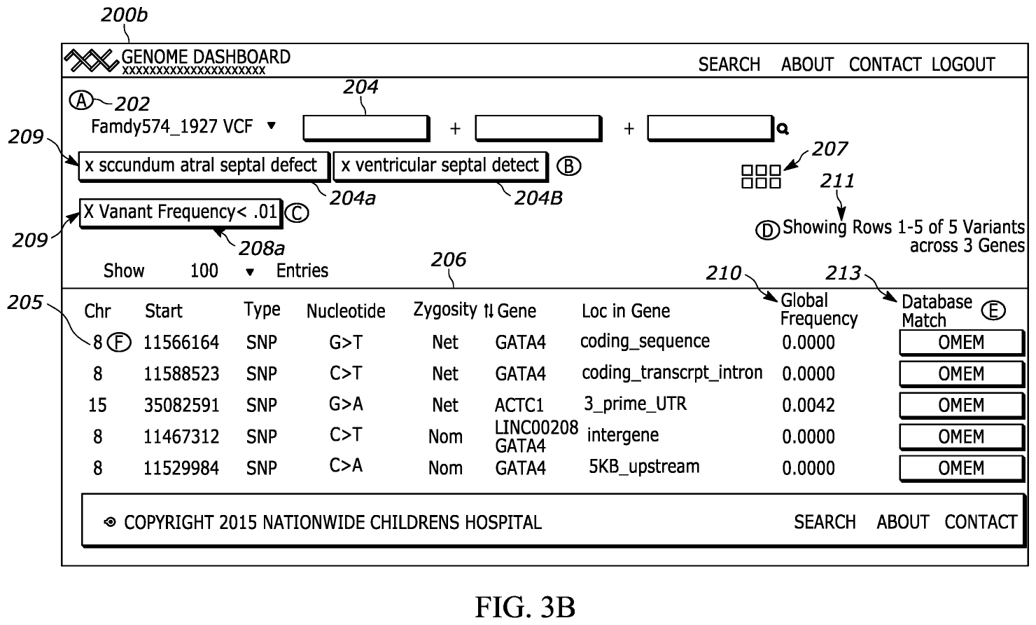

FIG. 3A-3B illustrate a second view 200b of the Genome Dashboard's web interface 200 including displaying results 216 of a sequencing data, clinical notes 208, and/or phenotype keyword search 204. The second view 200b illustrates: the sequencing data 202 that was uploaded; provides phenotype keyword upload locations 204; illustrates already uploaded phenotype keywords 204a, 204b; filter options 207 and/or a plurality of columns 206. The plurality of columns 206 illustrating the results 216 of a particular search, wherein the results are altered based upon the addition or subtraction of search parameters, filters, inputs, etc. In the illustrated example embodiment, the sequencing data 202 is illustrated as uploaded (e.g., an identifying element is present in the genome dashboard 200). In one example embodiment, a phenotype-driven search is performed using phonotype keywords 204 from the patient's clinical data 208, disease diagnoses, and/or phenotype or disease description 112. In the illustrated example embodiment, additional phenotype keywords 204a, 204b have been uploaded (e.g., the genome dashboard 200 illustrates the previously uploaded phenotype keywords), the additional phenotype keywords 204a, 204b are removable through a selection of a removal icon 209.

In the illustrated example embodiment of FIG. 3B, the filter option 207 is selected, wherein the selected filter is <1% population frequency. The filter selection 208a is illustrated and removable through a selection of the removal icon 209. The genome dashboard 200 utilizes inputs from the filter 207, and/or the phenotype keywords 204 to create edited phenotypes for a patient. In one example embodiment, the edited phenotype is generated by refining terms, adding new clinical data, adding a ‘must have’, ‘cannot have’ and/or other logical operators. In an example embodiment, the genome system 100 comprises term entry tools, such as, for example, type-ahead logic to propose terms based on the text entered, spell check, etc.

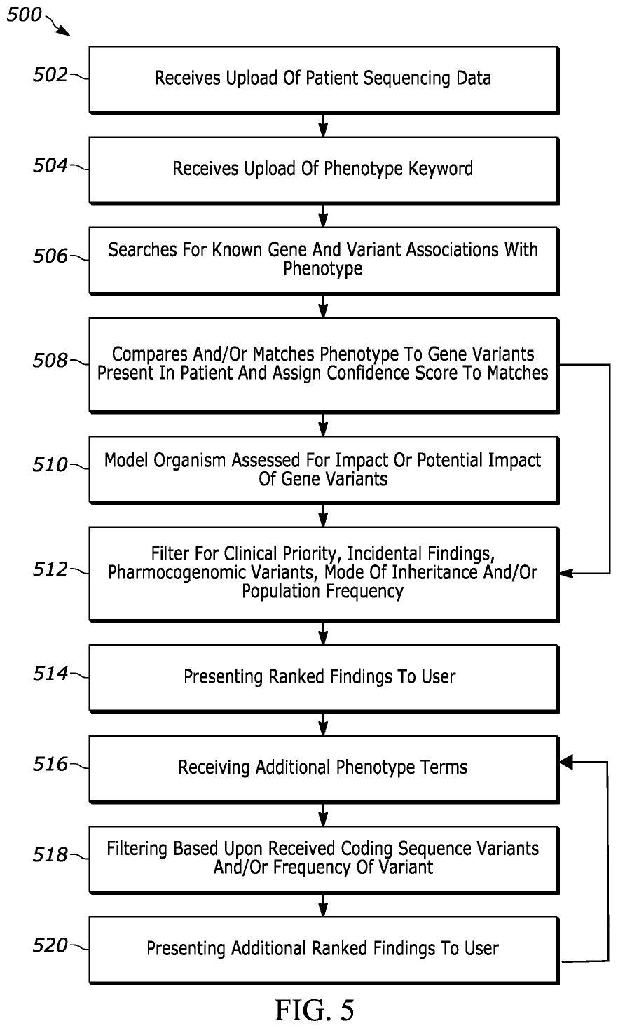

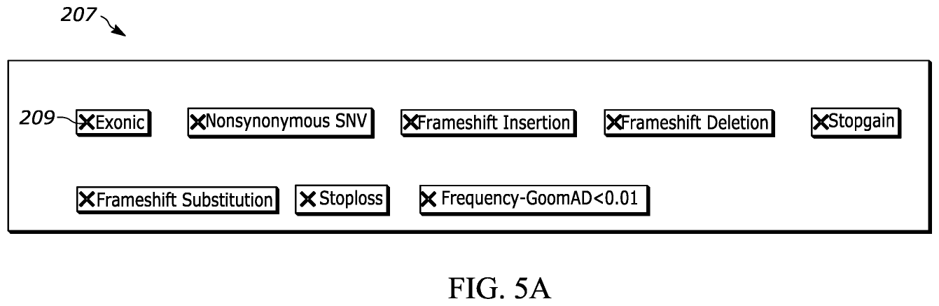

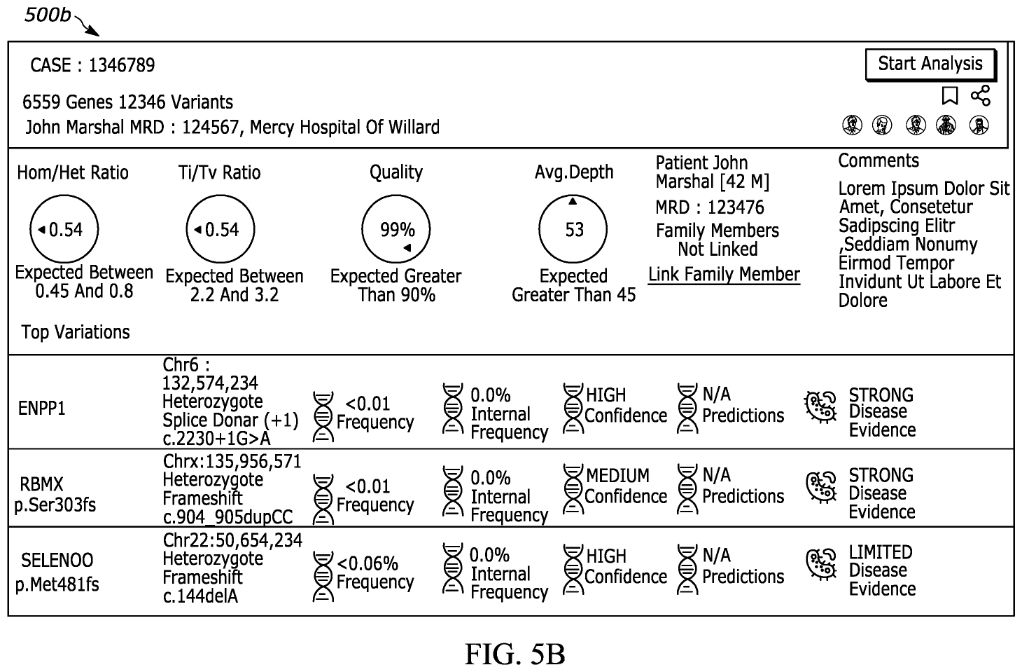

The genome dashboard 200 provides input options to add priority measures (e.g., such as 1-10) to increase or reduce the contributions of different phenotype terms that an Artificial Intelligence engine 602 will process (see FIGS. 5-7). The genome dashboard 200 provides input options to tag phenotype terms individually as ‘anomaly’ or in pairs/groups as ‘contradiction’. The genome dashboard 200 will be display in the user interface 206 the phenotype tag, as well as input the phenotype tag into the artificial intelligence engine 602, to facility learning by the artificial intelligence engine, and to sort results. To generate confidence annotations (e.g., likelihood of a match) to be added to phenotype or other terms, the artificial intelligence engine 602 and/or a user may use, for example, Evidence & Conclusion Ontology (ECO), as shown in described in ECO, the Evidence & Conclusion Ontology: community standard for evidence information Giglio M, Tauber R, Nadendla S, Munro J, Olley D, Ball S, Mitraka E, Schriml L M, Gaudet P, Hobbs E T, Erill I, Siegele D A, Hu J C, Mungall C, and Chibucos M C. (2018). Nucleic Acids Research, incorporated by reference in its entirety for all purposes. In one example embodiment, the artificial intelligence engine 602 utilizes ECO to add scientific evidence annotations and use Confidence Information Ontology to add annotations about the user's confidence in each annotation.

The genome dashboard 200 illustrates variants and/or genes 205 matching the search 211, wherein each variant or gene is illustrated within a row. In the illustrated example embodiment of the second view 200b, one of the plurality of columns 205 includes one or more links to external public databases. The external public databases are identified and presented to a user, wherein the links are matched based upon the phenotype or disease description 112. In one example embodiment, the links 213 include information about other individuals with similar disease and/or gene variations.

In the illustrated example embodiment of the second view 200b, one of the plurality of columns 206 includes identified variants (e.g., in a protein-coding sequence region of a gene). In the illustrated example embodiment of the second view 200b, additional columns of the plurality of columns 206 include chromosome numbers, start, type of variation, zygosity, gene, Loc in gene, global frequency 210, and/or database matches 213. In one example embodiment, the additional columns of the plurality of columns 206 are filterable.

In one example embodiment, in the second view 200b, responsive to the user selecting a confirmation mode to confirm or reject a clinical diagnosis, the genome dashboard 200 will output a Yes/No/Maybe/Partial confirmation, and/or a confidence score. In another example embodiment, in the second view 200b, responsive to the user selecting a primary diagnosis mode, the genome dashboard 200 will output top clinical recommendations that supports the phenotype and genomic data, as identified and ranked by the knowledge base 108.

In yet another example embodiment, in the second view 200b the genome dashboard 200 will, responsive to the user selecting a secondary analysis mode, output additional variants/diagnosis recommendations and hide the top clinical recommendations that support the phenotype and genomic data, as identified and ranked by the knowledge base 108. In yet another example embodiment, in the second view 200b the genome dashboard 200 will, responsive to the user selecting a genomic reinterpretation or phenotypic updates mode, identify recent changes in the reference databases (e.g., new knowledge) and output the recent changes as patient conditions to highlight any changes in interpretation based upon the recent changes.

Additionally, in an example embodiment, the genome system 100 will integrate additional clinical information (e.g., lab test results, blood work, physical presentations of illness, etc.) and additional genomic data (e.g., proteomics, epigenomics, histology, etc.) in order to better filter the data for the second view 100b (e.g., a diagnosis confirm/reject or diagnosis recommendation). The genome system 100 will generate pop-ups and reports that illustrate how selected gene variants connect to the phenotype or disease description 112 and/or to a proposed diagnoses. The genome system 100 will illustrate high priority mismatches between genomic interpretations and phenotype or disease description 112. Another report will show in a filterable list of all gene variants connected by the genome system 100 with a particular phenotype or disease description 112. If there are differences between the canonical disease/genomics the genome system 100 will highlight the differences with visual indicators.

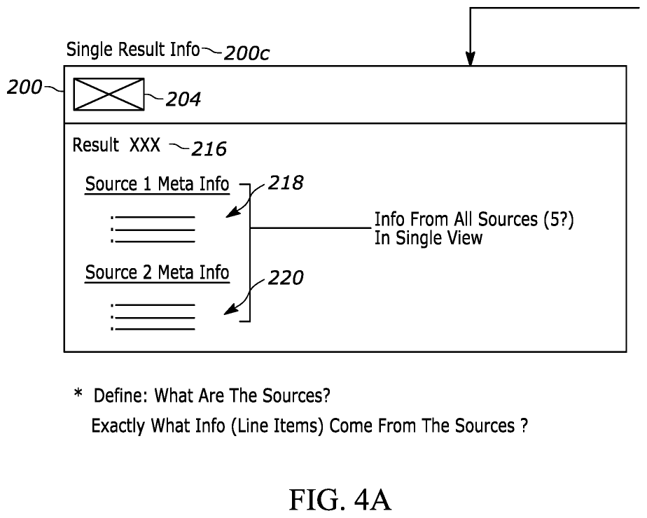

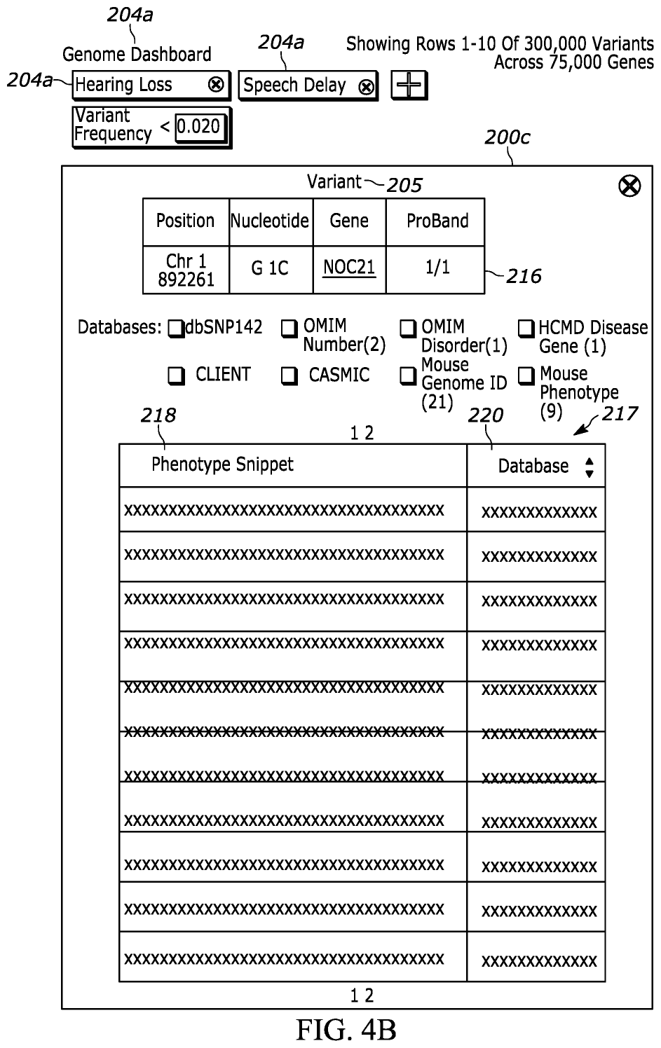

FIG. 4A-4B illustrate a third view 200c of the genome dashboard's web interface 200. The third view 200c illustrates additional phenotype information based upon a selection of a variant 205 identified in the second view 200b. The third view 200c illustrates the selected variant 205 and results 216 related to that variant, as well as databases searched 217. In the illustrated example embodiment, a second set of columns 218 illustrates an identified phenotype snippet 218 and a particular database 220 from which the snippet was extracted. Each snippet 218 and database 220 pair are presented in a row.

Illustrated in FIG. 5 is a method 500 of utilizing the genome system 100 to generate ranked findings to a user. At 502, the genome system 100 receives an upload of a patient's sequencing data 202. At 504, the genome system 100 receives an upload of one or more phenotype or disease descriptions 112 (e.g., either directly entered as phenotype keywords 204 and/or identified/parsed from clinical notes 208). In one example embodiment, the genome system 100 identifies/parses the one or more phenotype or disease descriptions 112 using standard phenotype and standard disease ontologies. Wherein standard phenotype and standard disease ontologies include default methodologies, such as Human Phenotype Ontologies (HPO). At 506, the genome system 100 searches for and identifies known genes and/or gene variants that are associated with one or more of the phenotype or disease descriptions 112. At 508, the genome system 100 compares and/or matches one or more of the phenotype or disease descriptions 112 to gene variants that are present in the sequencing data 202 to generate identified gene variants. In one example embodiment, the genome system 100 compares or matches in step 508 based upon the ontologies identified/parsed from the clinical notes 208 and phenotypes and disease variants identified at step 506. In one example embodiment, the genome system 100 compares or matches in step 508 based upon scoring relationships of the various one or more of the phenotype or disease descriptions 112 to gene variants

At 510, in an optional step, the genome system 100 assesses one or more model organisms for impacts or potential impacts of the identified gene variants present in the sequencing data. In one example embodiment, the one or more model organisms are identified human orthologs that are maintained within the knowledgebase 108. In another example embodiment, the model organisms are identified using an external knowledgebase.

At 512, the genome system 100 filters for clinical priority, incidental findings, pharmocogenomic variants, mode of inheritance, and/or population frequency. For example, illustrated in the example embodiment of FIG. 5A, applied filters 207 are illustrated, wherein the applied filters are removable via the removal icon 209. In one embodiment, the genome system 100 selects the filters, in another embodiment, the filters are input by a user. In this example, the user inputs filters and steps 508-514 are repeated iteratively, as filters are input or removed. At 514, the genome system 100 presents ranked findings to the user based upon the confidence score assigned and/or the filter 207 used. At 516, the genome system 100 receives additional phenotype or disease descriptions 112 (e.g., the user is adding terms, the user has added to clinical notes about said patient, etc.).

At 518, the genome system 100 filters the additional phenotype terms based upon additional filters, including received coding sequence variants and/or frequency of variant. In one example embodiment, the additional filters, such as population frequency, clinically relevant variants, etc. are available from the knowledgebase 108 and may be applied into scoring for matching variants to phenotypes. In one example embodiment, the genome system 100 will rank a variant identified as clinically relevant (e.g., having a higher association with a disease phenotype) higher than a variant that is not associated with clinical outcomes (e.g., the variant has a low, or no association with a disease phenotype), where higher ranking indicates greater likelihood of the phenotype being associated with the variant. In one example embodiment, the variant is identified as relevant if it has an association with a phenotype or disease description 112 received from the clinical notes and/or the user over a variant association threshold. At 520, the genome system 100 presents additional ranked findings 500b to the user based upon the additional phenotype or disease descriptions 112 and/or the additional filters (see, for example, FIG. 5B). In one example, steps 516-520 are iteratively repeated as additional phenotype terms become available and/or additional filtering is input.

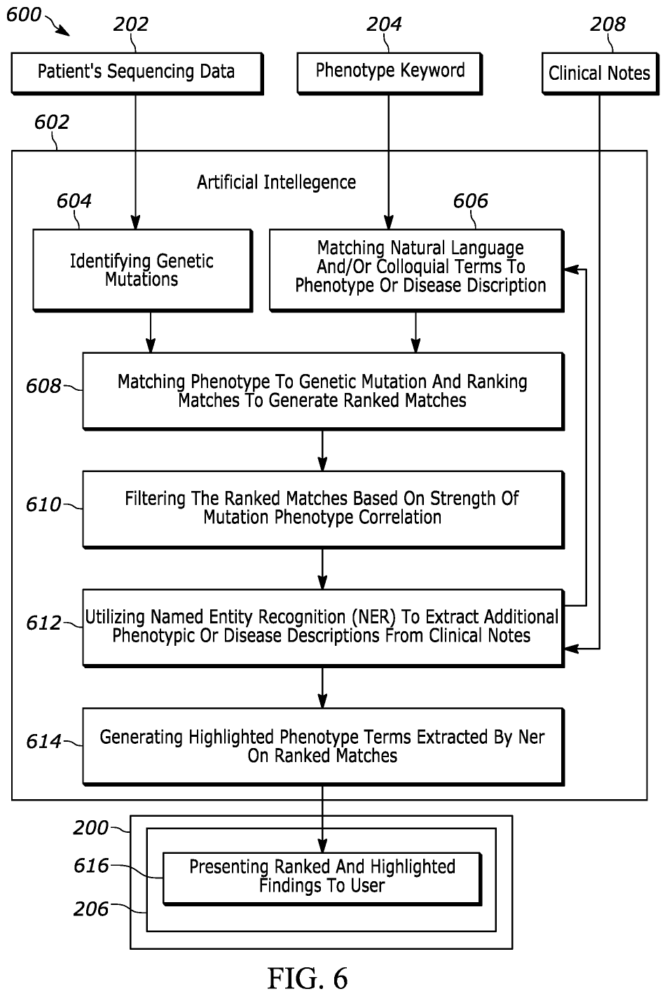

Illustrated in FIG. 6 is a method 600 of using the genome dashboard 200, including inputs and outputs utilized in presenting ranked and highlighted findings to a use. In one example embodiment, findings are ranked and highlighted based upon the artificial intelligence engine 602 selecting and utilizing one or more filters. In this example embodiment, the ranking and highlighting is generated through the use of filters such as level of pathogenicity (e.g., performed at a population level), type of mutations associated with an identified variant (e.g., protein missense mutations), clinical significance of the identified variant, allele/population frequency of the identified variant, and/or phenotype matching between patient's phenotype keyword 204, clinical notes 208, and known phenotype/disease associations between identified variant and/or the patient's phenotype keyword 204 and/or clinical notes 208. In the illustrated example embodiment, the artificial intelligence engine 602 receives at least one of the patient's sequencing data 202, phenotype keywords 204, and/or clinical notes 208. At 604, the artificial intelligence engine 602 identifies genetic mutations present in the patient's sequencing data 202. At 606, the artificial intelligence engine 602 matches natural language and/or colloquial terms to the phenotype or disease descriptions 112. The artificial intelligence engine 602, comprising a natural language processing (NLP) engine, utilizes standard phenotype and standard disease ontologies to build automatons to scan clinical notes 208. Further, in some example embodiments, the artificial intelligence engine 602 utilizes reinforcement learning based on pre-trained medical models, such as Medical-BERT, to create phenotype and disease Named-Entity-Recognizers that matches natural language and/or colloquial terms to the phenotype or disease descriptions 112. At 608, the artificial intelligence engine 602 matches the phenotype or disease descriptions 112 to the genetic mutation and ranks the matches of the phenotype or disease descriptions 112 to the genetic mutation from best to worst. In one example embodiment, the phenotype or disease descriptions 112 to the genetic mutation relationship is ranked by assigning a confidence score to the match, wherein a highest confidence score is a best match. In one example embodiment, the confidence score is based on a context of the filters, wherein the ranking is based on a combination of filters. In this example embodiment, each filter that the artificial intelligence engine 602 utilizes is normalized over a range from 0 to 1, wherein the confidence score is determined based upon the normalized filter values. In one example embodiment, a normalized filter value of 0 is no confidence and 1 is 100% confidence. Responsive to the user of multiple filters, multiple normalized filter values are generated and combined to generate the confidence score.

At 610, the artificial intelligence engine 602 filters the ranked matches based upon a strength of mutation/phenotype correlation. The artificial intelligence engine 602 performs the mutation/phenotype correlation, and subsequently performs additional mutation/phenotype correlations based upon one or more filters a user may emphasize or deemphasize. In one example, the user emphasizes a filter by providing a weighting/priority score that will be utilized to rank matches. In this example embodiment, the artificial intelligence engine 602 calculates a composite score based upon the strength of the mutation/phenotype correlation, additional mutation/phenotype correlations, and/or user provided weighting/priority scores.

In one example embodiment, the user provided weighting/priority scores are generated where a user determines that some of the phenotypes are more/less important than others for the patient or a specific disease. The user is provided with an option, by the artificial intelligence engine to weight the contributions of identified phenotypes from along a value scale (e.g., 1-5). Further, wherein the user is not confident that the patient was diagnosed correctly among similar phenotypic elements the user is provided with the option to alter the weighting to reduce the contribution of those phenotypes that the user has less confidence. Likewise, the artificial intelligence engine 602 presents the user with the option to change the weighting of certain genome variants because the user believes the variant is very important to the diagnosis (e.g., raise the value from default 3 to 5), or because there is a lack of scientific evidence that the variant is important to a disease (e.g., lower the value lower it from 3 to 1) to reduce the impact of a mismatch on the diagnosis. In this example embodiment, the artificial intelligence engine 602 assigns a default weight (e.g., 3) to all variants, wherein the user has the option of altering such default weights.

At 612, the artificial intelligence engine 602 utilizes named entity recognition (NER) to extract additional phenotype or disease descriptions 112 from the clinical notes 208. In one example embodiment, at 606, the extracted additional phenotype or disease descriptions are translated from natural language and/or colloquial terms to phenotype or disease descriptions 112 (as described at step 606). The extracted additional phenotype or disease descriptions 112 undergo steps 608-610. At 614, the artificial intelligence engine 602 generates highlighted (e.g., visually differentiated) phenotype or disease descriptions 112 extracted by NER on the generated ranked and filtered findings. At 616, the artificial intelligence engine 602 presents the ranked and highlighted findings to the user on the user interface 206 of the genome dashboard 200.

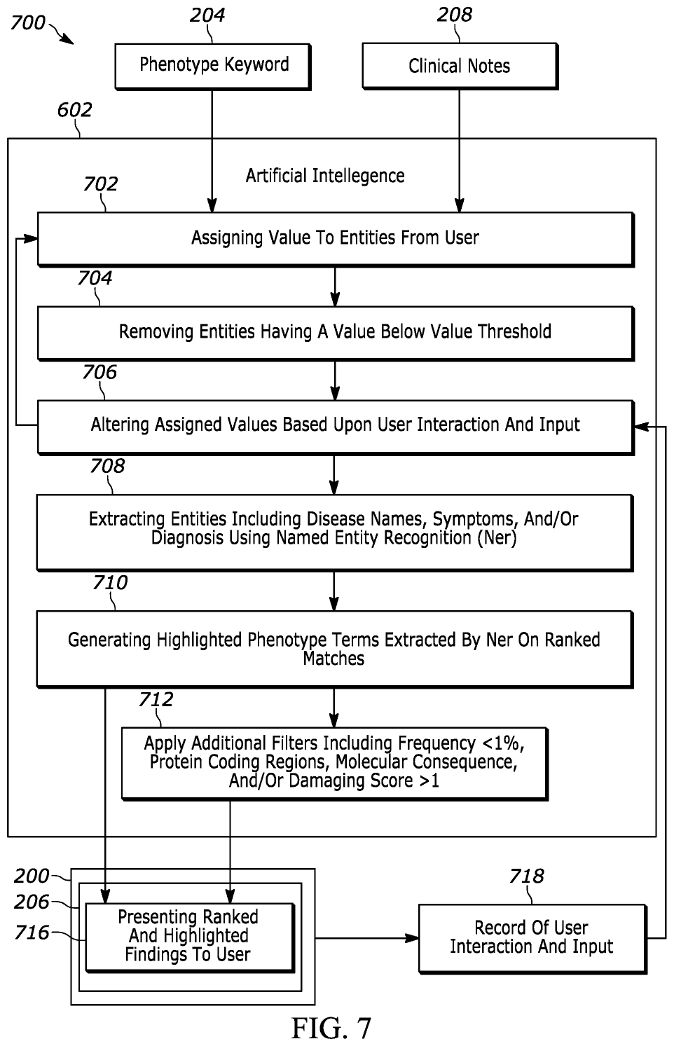

Illustrated in FIG. 7 is a method 700 using the genome dashboard 200, including incorporating records of user interaction in generating ranked and highlighted findings to present a user. In the illustrated example embodiment, the artificial intelligence engine 602 receives at least one of the phenotype keywords 204, and/or clinical notes 208. At 702, the artificial intelligence engine 602 assigns value to entities (e.g., keywords 204, clinical notes 208, phenotype or disease descriptions 112, etc.) as they relate to the genome sequence 202 received from the user. At 704, the artificial intelligence engine 602 removes entities having an assigned value below a value threshold (e.g., that do not have significant phenotype/gene variant match). At 706, the artificial intelligence engine 602 alters the assigned values based upon user interaction with various entities and user input responsive to results using previously assigned values, as well as additional information added by the user, and/or additional relationships discovered between a particular phenotype/gene variant pair. Steps 702-704 are repeated when new values are assigned. Stated another way, the artificial intelligence engine 602 first identifies phenotypes based on NLP extraction from the clinical notes 208. The user may add or delete phenotypes based on the user's clinical knowledge (e.g., utilizing phenotype keywords 204) that further modifies the ranking of the genetic variants. In one example embodiment, the user selects filters are utilized by the artificial intelligence engine 602, as well as assigning the weighting/priority scores to the selected filters for determining different assigned values for ranking matches.

At 708, the artificial intelligence engine 602 extracts entities, including disease names, symptoms, and/or diagnosis using NER. At 710, the artificial intelligence engine 602 generates highlighted (e.g., visually differentiated) phenotype terms extracted by NER on a generated ranked match (e.g., such as the ranked match generated at 614 of method 600). At 712, in an optional step, the user applies additional filters including frequency <1%, protein coding regions, molecular consequence, and/or damaging score>1. At 716, the artificial intelligence engine 602 presents the ranked and highlighted findings to the user on the user interface 206 of the genome dashboard 200. At 718, the artificial intelligence engine 602 records the user interaction with the ranked finding based upon the entities and user input responsive to results using the currently assigned values. In one example embodiment, the user interaction is utilized to alter the assigned value in step 706.

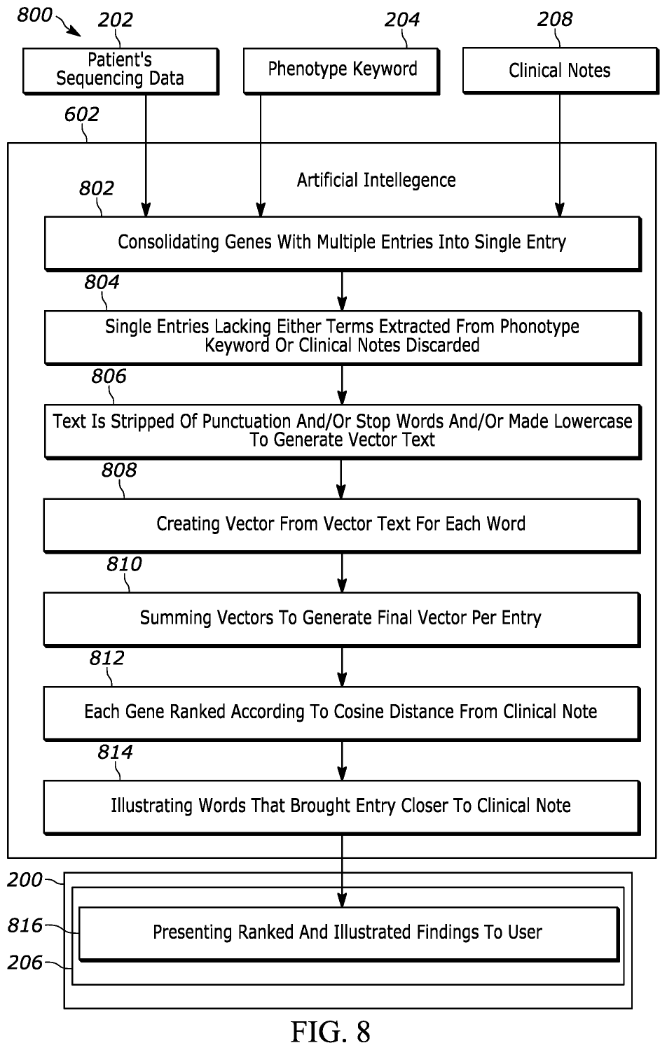

Illustrated in FIG. 8 is a method 800 of using the genome dashboard 200, including generating ranked and illustrated findings from multiple inputs, including plain text inputs. In the illustrated example embodiment, the artificial intelligence engine 602 receives at least one of the patient's sequencing data 202, phenotype keywords 204, and/or clinical notes 208. At 802, the artificial intelligence engine 602 consolidates genes with multiple entries into a single entry. Stated another way, responsive to identifying a patient that shows multiple variants within a single gene or heterozygous alleles, the artificial intelligence engine 602 consolidates the multiple variants together under a single gene/column 205 (see FIG. 3A). At 804, the artificial intelligence engine 602 discards any single entry that lacks either terms extracted from the phenotype keywords 204, and/or clinical notes 208 (e.g., generating phenotype or disease descriptions 112). At 806, the artificial intelligence engine 602 strips text present in the phenotype keywords 204, and/or clinical notes 208 of punctuation and/or stop words and generates vector text by putting all terms in lower case text. At 808, the artificial intelligence engine 602 creates a vector from the vector text for each identified word. At 810, the artificial intelligence engine 602 sums the vectors to generate a final vector for each entry. At 812, the artificial intelligence engine 602 ranks each gene according to a cosine distance from an identified phenotype or disease description 112. At 814, the artificial intelligence engine 602 illustrates (e.g., visually differentiates) words that brought entities closer to the identified phenotype or disease description 112. At 816, the artificial intelligence engine 602 presents the ranked and highlighted findings to the user on the user interface 206 of the genome dashboard 200.

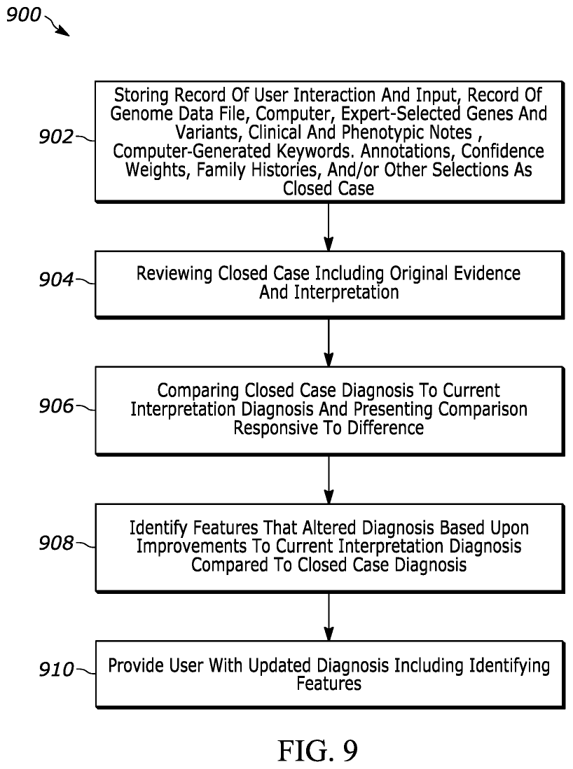

Illustrated in FIG. 9 is a method 900 of utilizing the genome system 100 including incorporating records of user interaction into providing a diagnosis. At 902, the genome system 100 stores records of user interaction and input, gene sequence 202, computer and/or expert-selected genes and variants, clinical and phenotypic notes, computer-generated keywords and/or annotations, confidence weights, family histories, and/or other selections as a closed case. At 904, the genome system 100 reviews closed cases including reviewing original evidence and interpretation (e.g., diagnosis reached, gene variant identified as significant, etc.). At 906, the genome system 100 compares the closed case diagnosis to a current interpretation diagnosis (e.g, using new findings, new user inputs, etc.), and presents the comparison to the user responsive to the presence of a difference between the closed case diagnosis and the current interpretation diagnosis. For example, responsive to patient consent for automatic notice to the user, new updates to clinical variant analysis reference databases are incorporated into the genome system 100. The genome system 100 will identify/change the interpretation/diagnosis for a patient based upon the new updates and a newly established clinical importance of genes present in the genome sequence 202. Additionally, the genome system 100 will obtain data from the knowledge base 108 as it is updated (updated values) to reflect the current information around phenotypes, diseases, and the clinical significance of gene variants. In one example embodiment, updated values influence ranking of patient genetic variants, and thus influence potential diagnosis for a patient. In this embodiment, if a new diagnosis for a patient is found, a notification is sent to the user on the genome dashboard 200.

The genome system 100 provides notice to the user that a new analysis and new interpretation/diagnosis is available. At 908, the genome system 100 identifies which features altered the diagnosis (e.g., phenotype gene matching, gene association with diagnosis, etc.), for example based upon improvements to current diagnosis compared to closed case diagnosis. At 910 the genome system 100 provides the user with an updated diagnosis, including identifying the features that altered the updated diagnosis.

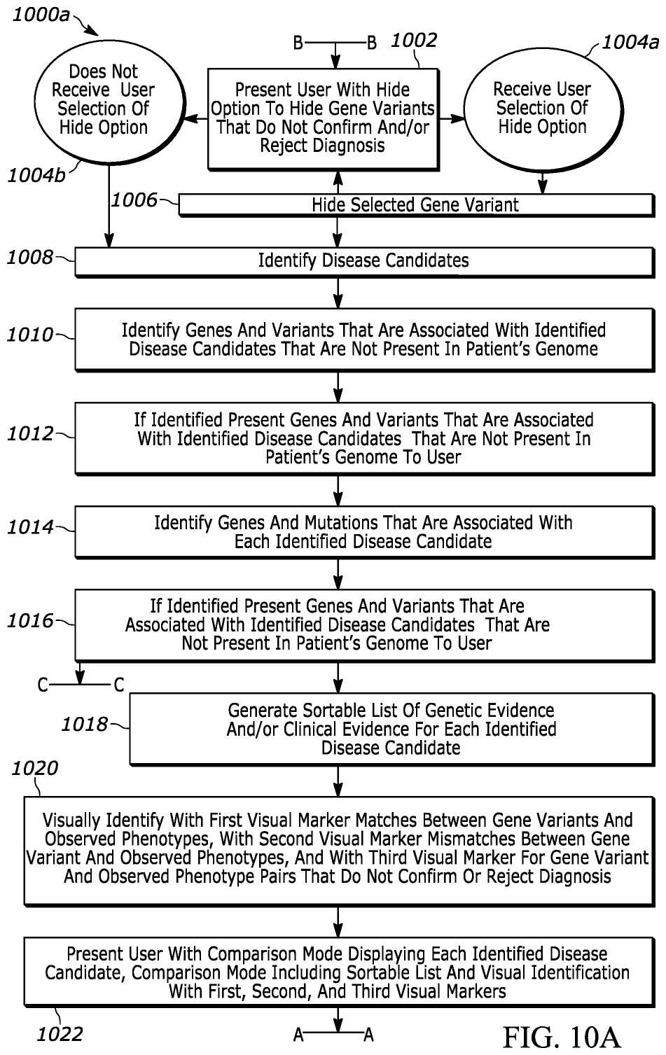

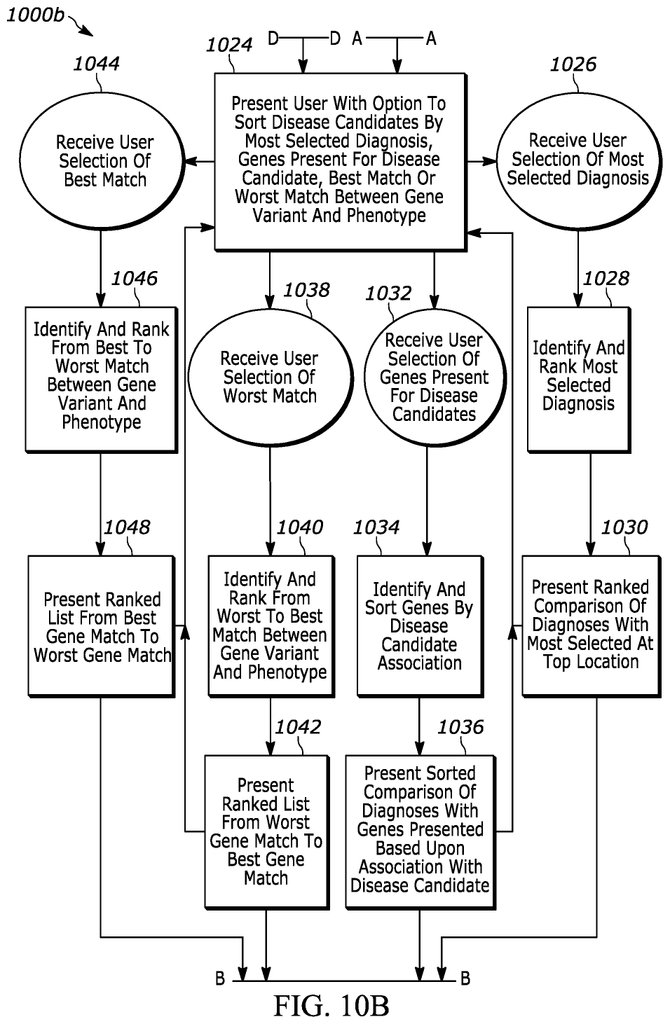

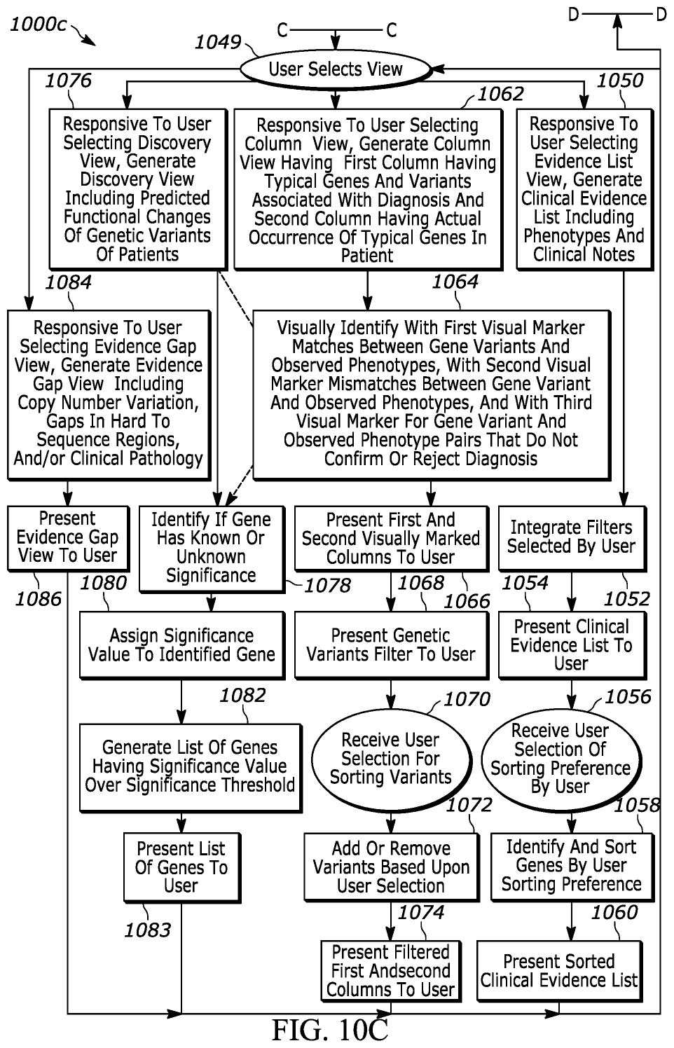

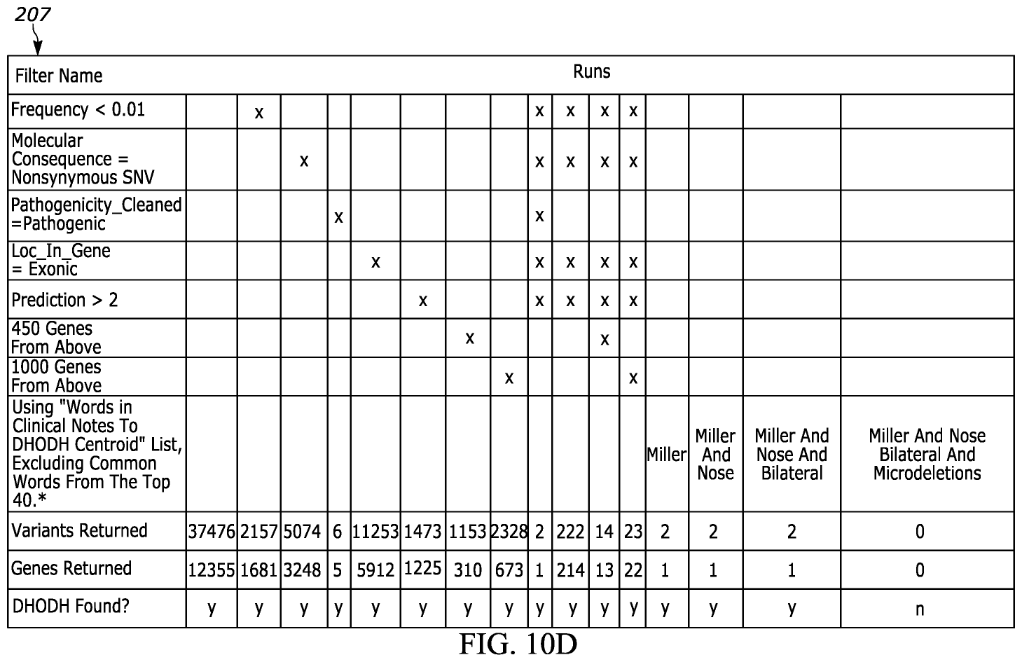

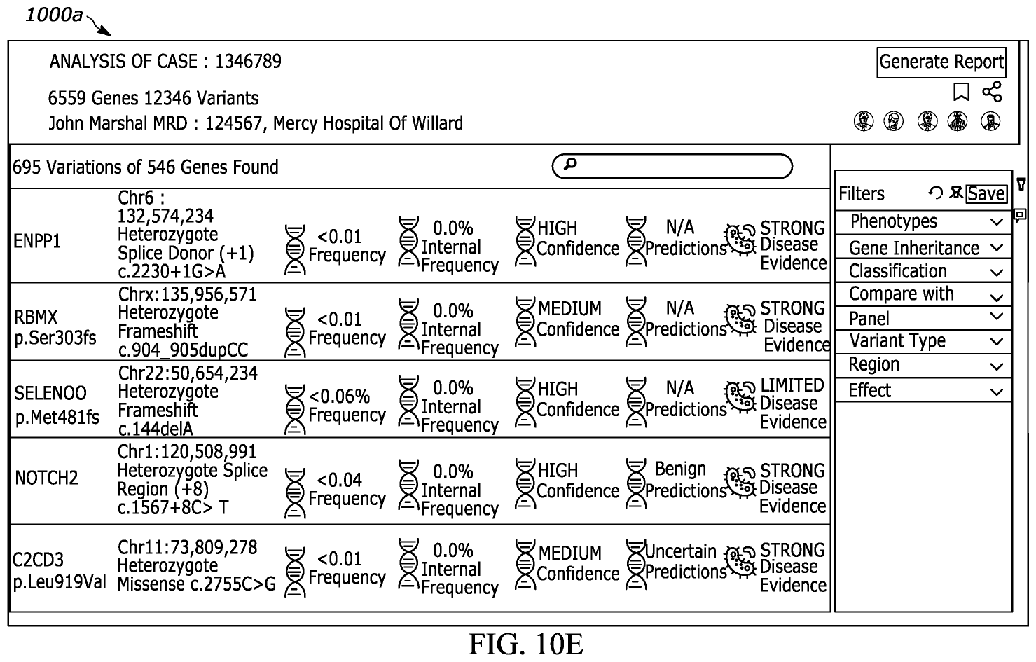

Illustrated in FIG. 10A is a method 1000a of utilizing the genome system 100 including generating a comparison mode display. At 1002, the user is presented with an option to hide gene variants that do not confirm or reject a diagnosis. At 1004a, the genome system 100 receives a user selection to hide the gene variant. At 1006, responsive to the selection of hide, the selected gene variant is hidden (e.g., such as when the user does not want to include that variant in the diagnosis). At 1004b, the genome system 100 receives no user selection to hide the gene variant, the gene variant is not hidden. At 1008, the genome system 100 identifies disease candidates. At 1010, the genome system 100 identifies genes and variants that are associated with the identified disease variant that are not present in the patient genome sequence 202. At 1012, the genome system 100, responsive to identifying non-present genes and variants, presents those genes and/or variants to the user. At 1014, the genome system 100 identifies genes and variants that are associated with the identified disease candidate in the genome sequence 202. At 1016, the genome system 100, responsive to identifying present genes and variants, presenting those genes and/or variants to the user. At 1018, the genome system 100 generates a sortable list of genetic evidence and/or clinical evidence for each identified disease candidate. At 1020, the genome system 100 visually identifies matches between gene variants and observed phenotypes with a first visual marker (e.g., green highlighting), mismatches between gene variants and observed phenotypes with a second visual marker (e.g., red highlighting), and gene variants and observed phenotype pairs that do not confirm or reject diagnosis with a third visual marker (e.g., yellow highlighting). At 1022, the genome system 100 presents user with a comparison mode, which display each identified disease candidate. In one example embodiment, the comparison mode includes generating a sortable list and visual identification with first, second, and third visual markers.

As continued in example method 1000b in FIG. 10B, continued from section line A-A, at 1024, the virtual reality system 100 presents the user with an option to sort disease candidates by most selected diagnosis, genes present for disease candidate, best match between gene variant and phenotype and worst match between gene variant and phenotype. At 1026, the user selects the most selected diagnosis option. At 1028, responsive to the user selecting the most selected diagnosis option, the genome system 100 identifies and ranks the most selected diagnosis. At 1030, the genome system 100 presents a ranked comparison of the identified most selected diagnosis with the most selected at a top or most prominent location. At 1032, the user selects the gene present for disease candidate option. At 1034, responsive to the user selecting the gene present for disease candidate option, the genome system 100 identifies and sorts the genes based upon confidence that gene is present for the disease candidates. At 1036, the genome system 100 presents sorted comparisons of the genes present for each disease candidate with the highest confidence score gene disease candidate pair at a top or most prominent location. At 1038, the user selects the worst match option. At 1040, responsive to the user selecting the worst match option, the genome system 100 identifies and sorts the genes based upon the worst match between the gene variant and the phenotype. At 1042, the genome system 100 presents a sorted ranked list from worst gene match to best gene match, with the worst gene phenotype pair at a top or most prominent location. At 1044, the user selects the best match option. At 1046, responsive to the user selecting the best match option, the genome system 100 identifies and sorts the genes based upon the best match between the gene variants and the phenotypes. At 1048, the genome system 100 presents a sorted ranked list from best gene match to worst gene match, with the best gene phenotype pair at a top or most prominent location. As continued from 1030, 1036, 1042, and/or 1048 and section line B-B of example method 1000b and continued in example method 1000a in FIG. 10a from section line B-B, at 1002, the user may repeat steps 1002-1022.

Alternately, as continued from section line C-C extending from 1016 in FIG. 10A and continued from section line C-C in example method 1000c at 1049, the virtual reality system 100 presents the user with a view option. Alternately, the genome system 100 selects the view option for the user, based upon the search being performed, past user interactions, the gene sequence 202 input, the phenotype keyword 204 input, the clinical notes 208 input, or the like.

At 1050, responsive to the user selecting an evidence list view, the genome system 100 generates a clinical evidence list including phenotypes 204, clinical notes 208, and/or phenotype or disease description 112. At 1052, the genome system 100 integrates filters 207 selected by the user. In one example, FIG. 10d illustrates a plurality of filters 207 that a user may utilize. At 1054, the genome system 100 presents the clinical evidence list to the user. At 1056, the genome system 100 receives a selection of a sorting preference generated by a user. At 1056, responsive to the user selecting a sorting preference, the genome system 100 identifies and sorts the clinical evidence list based upon the sorting preference. At 1060, the genome system 100 presents the sorted clinical evidence list 1000e to the user (see, for example, FIG. 10e).

At 1062, responsive to the user selecting column view, the genome system 100 generates a column view including a first column having typical genes and variants associated with diagnosis and a second column having actual occurrence of typical genes in the gene sequence 202 of the patient. At 1064, the genome system 100 visually identifies matches between gene variants and observed phenotypes with a first visual marker, mismatches between gene variants and observed phenotypes with a second visual marker, and gene variants and observed phenotype pairs that do not confirm or reject diagnosis with a third visual marker. At 1066, the genome system 100 presents first and second visually marked columns to the user. At 1068, the genome system 100 presents a genetic variants filter to the user. At 1070, the genome system 100 receives a user selection for sorting variants. At 1072, the genome system 100 responsive to receiving a selection for sorting variants, adds or removes variants based upon the user selection. At 1074, the genome system 100 presents the filtered first and second columns to the user.

At 1076, responsive to the user selecting discovery view, the genome system 100 generates a discovery view including predicting functional changes/consequences of genetic variants of a patient. The functional consequences of the genetic variants are predicted by annotating the patients genome sequence with functional annotators that generate annotations associated with various genetic variants. In an example embodiment, the functional annotators include example functional annotators such as JANNOVAR and Exomiser. The annotations are extracted from the patient's genome sequence and assigned values for use in the ranking of genetic variants.

Optionally, the genome system 100 adds visual indicators as in step 1064 to the discovery view. At 1078, the genome system 100 identifies if a gene has a known or unknown significance. At 1080, the genome system 100 assigns a significance to a gene if known. At 1082, the genome system 100 generates a list of genes having an assigned significance over a significance threshold. Genes having an assigned significance under the significance threshold are not presented to the user. At 1083, the genome system 100 presents the list of significant genes to the user.

At 1084, responsive to the user selecting evidence gap view, the genome system 100 generates an evidence gap view including copy number variation, gaps in hard sequence regions and/or clinical pathology. At 1086, the genome system 100 presents the evidence gap view to the user. The evidence gap view will also inform the user when there is additional genomic data not present that could help confirm or reject a specific diagnosis. The additional genomic data comprises copy number variation (CNV), genotyping, sequencing a genomic region beyond Whole Exome Sequencing (e.g., if that was a filter), and/or other chromosomal aberrations (larger insertions, deletions, recombinations). The user may select the evidence gap view at any point of interaction with a case in the genome dashboard 200, as such the user can add additional genomic data for the patient at any time during the diagnosis.

As continued from 1060, 1074, 1083, and/or 1086 and section line D-D of example method 1000c and continued in example method 1000b in FIG. 10b from section line D-D, at 1024, the user may repeat steps 1024-1048. It would be understood by one having ordinary skill in the art after reviewing this disclosure and associated figures that the steps of method 1000a-1000c can be completed in different orders, and options may be presented, be constantly present, be accessible to a user via a search bar, or the like.

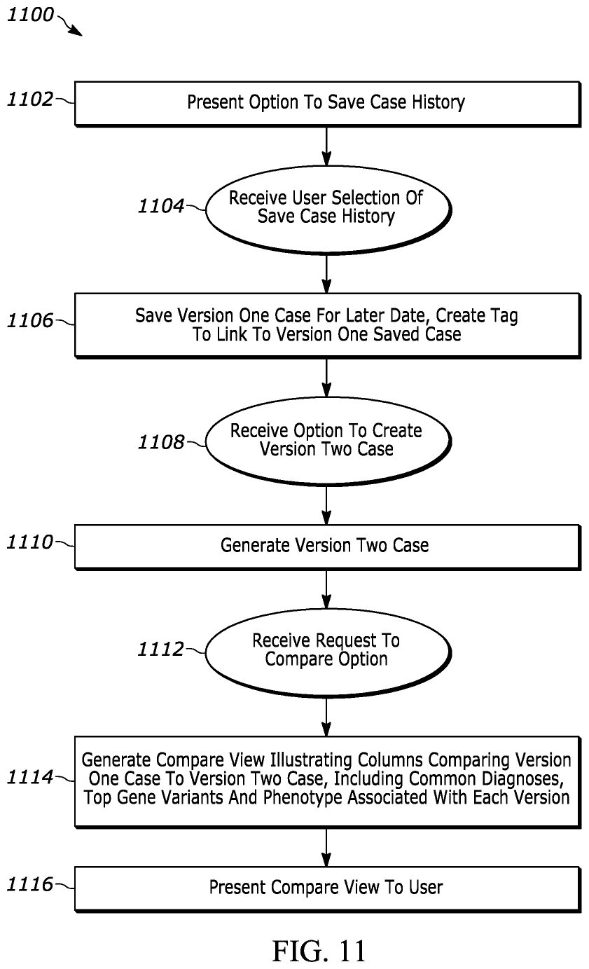

Illustrated in FIG. 11 is a method 1100 of utilizing the genome dashboard 200 of the genome system 100 to generate one or more versions of a case and comparing them. At 1102, the genome dashboard 200 presents a save case history option to the user to save a case history. At 1104, the genome system 100 receives a user selection of the save case history option. At 1106, responsive to receiving the user selection of the save case history option, the genome system 100 saves a version one case for a later date and creates a tag to link to the version one case. The tag is added to an archive of key genomic variants and key genomic interpretations for each case to enable the user to retrieve the version one case (e.g., an initial diagnosis). At 1108, the genome system 100 receives a user selection of the create a version two case. At 1110, responsive to receiving the user selection of the save case history option, the genome system 100 generates a version two of the case. In one example embodiment, the version one case will not be closed and the user (same or new) can continue the version one case or add the version two case to create a parallel interpretation record (e.g., a second opinion). At 1112, the genome system 100 receives a user request to compare option. At 1114, responsive to receiving user request to compare option, the genome system 100 generates a compare view illustrating columns comparing the version one case and the version two case, including common diagnosis, top gene variants and phonotype associated with each version.

The version two case (e.g., the current diagnosis) is compared as either a reinterpretation or a full new diagnosis of the original sequence data 202 or a full new diagnosis using new clinical and new genomic data. In one example embodiment, visual indicators (e.g., highlights) are applied to what has changed in the patient phenotype input (if any), what has changed in the genomic data input (if any), what has changed in the key variant analysis, and/or what has changed in expert guided diagnosis recommendations. At 1116, the genome system 100 presents the compare view to the user on the genome dashboard 200. The compare view, the version one case, and/or the version two case are archived as soon as a case is closed. The genome system 100 enables the user to time/date stamp specific versions of a case, as well as to add and save specific annotations and bookmarks for genes, variants, phenotypes, included diagnoses, and/or excluded diagnoses. In one example embodiment, the version two case is created by selecting a new view option in order to save all the work that has been done on the version one case and then begin again with a reset case or retain the version one case work and make changes such as selecting or deselecting the genes/variants, by adding stars or ‘X’s to create annotations showing interest or disinterest (e.g., star indicates interest, and x indicates disinterest). The genome system 100 allows the user to compare one or more versions of a case for a particular patient side-by-side.

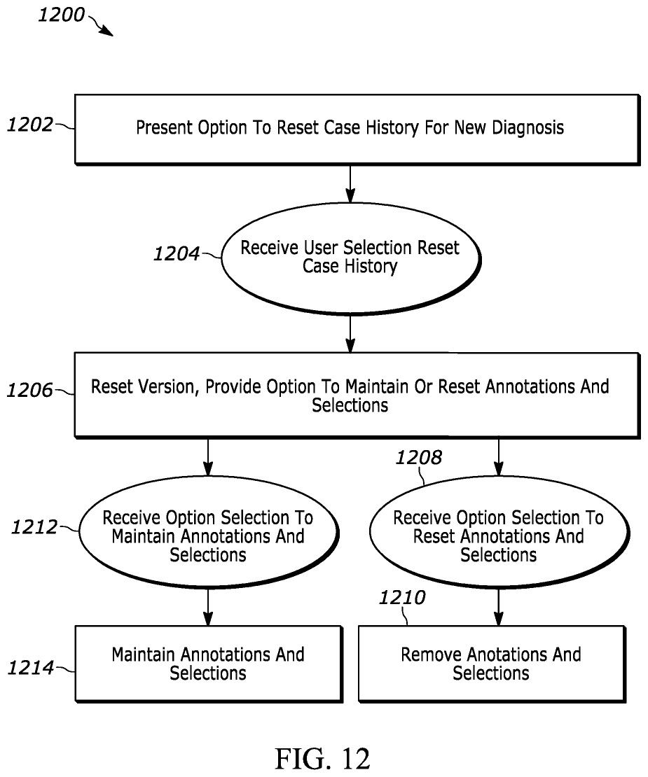

Illustrated in FIG. 12 is a method 1200 of utilizing the genome dashboard 200 of the genome system 100 to partially or completely reset a case history. At 1202, the genome dashboard 200 presents an option to reset case history for a new diagnosis. At 1204, the genome system 100 receives the user selection of the option to reset case history for a new diagnosis. At 1206, responsive to receiving the user selection of the option to reset case history for a new diagnosis, the genome system 100 resets the version and provides the user an option to maintain or reset annotations and selections (e.g., filters, notes, etc.). At 1208, the genome system 100 receives a user selection of the option to reset annotations and selections. At 1210, responsive to receiving the user selection of the option to reset annotations and selections, the genome system resets all annotations and selections. At 1212, the genome system 100 receives a user selection of the option to maintain annotations and selections. At 1214, responsive to receiving the user selection of the option to maintain annotations and selections, the genome system 100 maintains all annotations and selections with regard to further searches, genome sequence 204 inputs, clinical note 208 inputs, etc.

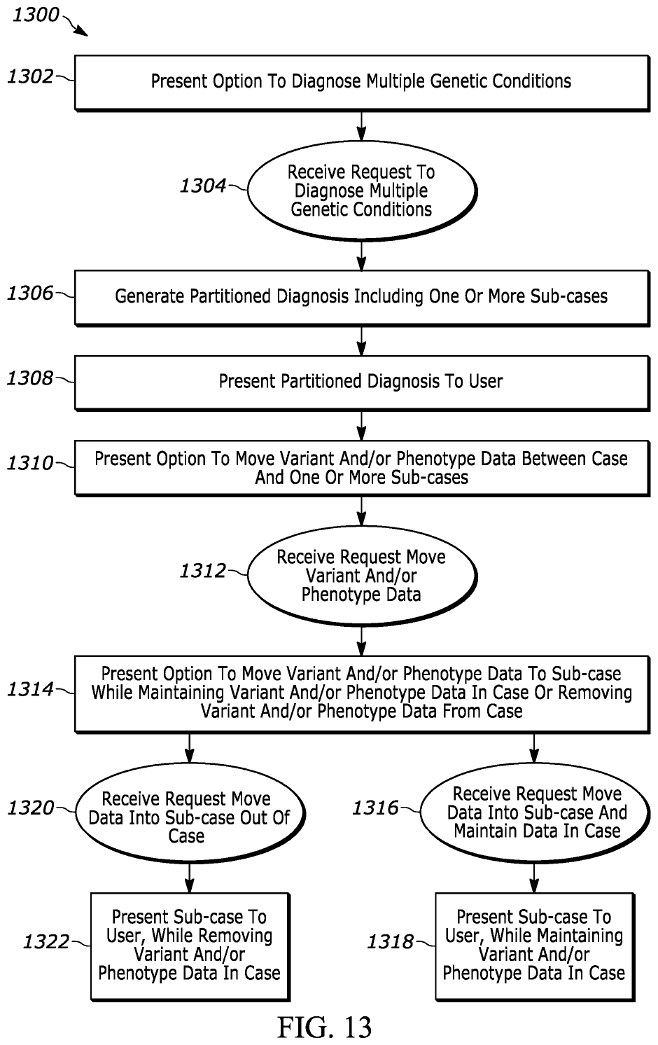

Illustrated in FIG. 13 is a method 1300 of utilizing the genome dashboard 200 of the genome system 100 including diagnosing multiple genetic conditions. At 1302, the genome dashboard 200 presents a diagnose multiple genetic conditions option to the user. At 1304, the genome system 100 receives request to diagnose multiple genetic conditions. At 1306, responsive to receiving the request to diagnose multiple genetic conditions, the genome system 100 generates a partitioned diagnosis including one more sub-cases.

At 1308, the genome system 100 presents the partitioned diagnosis, including one more sub-cases to the user on the genome dashboard 200. At 1310, the genome system 100 presents the option on the genome dashboard 200 to the user to move variant and/or phenotype data between a main case and one or more sub-cases. At 1312, the genome system 100 receives a request to move variant and/or phenotype data between a main case and one or more sub-cases. At 1314, responsive to receiving the request to move variant and/or phenotype data between a main case and one or more sub-cases, the genome system 100 presents an option to move the variant and/or phenotype data to the sub-case while maintaining the data in the main case, or to move the variant and/or phenotype data into the sub-case and out of the main case. At 1316, the genome system 100 receives a request to move variant and/or phenotype data from a main case to one or more sub-cases and maintain variant and/or phenotype data in the main case. At 1318, responsive to receiving the request to move variant and/or phenotype data from the main case to one or more sub-cases and maintain the variant and/or phenotype data in the main case, the genome system 100 presents the sub-case to the user including the selected variant and/or phenotype data, while maintaining the selected variant and/or phenotype data in the main case. At 1320, the genome system 100 receives a request to move variant and/or phenotype data from a main case to one or more sub-cases and remove the variant and/or phenotype data from the main case. At 1318, responsive to receiving the request to move variant and/or phenotype data from the main case to one or more sub-cases and remove the variant and/or phenotype data from the main case, the genome system 100 presents the sub-case to user including the selected variant and/or phenotype data, while removing the selected variant and/or phenotype data from the main case.

The genome dashboard 200 and genome system 100 offer an effective solution by allowing users to upload sequencing data and explore and compare against known gene-disease associations in other humans and closely related animal models. Comparing sequencing data to that of other humans is one of the best and most efficient methods to help identify gene variants responsible for human disease.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

To the extent that the materials for any of the foregoing embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A genome system for displaying an interactive genome dashboard, the genome system comprising:
a processing device having a processor configured to perform machine learning and performing a matching function between phenotype keywords and gene variants identified in a genome sequence to create gene matches based upon multiple text inputs and the genome sequence introduced through the interactive genome dashboard, the processing device comprising memory wherein previously generated matches are tagged and stored based upon the multiple text inputs, the genome sequence, and subsequent receipt of user interaction with the generated matches, the processing device:
receives one or more phenotype keywords and the genome sequence from the genome dashboard;
identifies genetic variants associated with the phenotype keyword;
matches the genetic variants to known genetic variants to generate a first diagnosis;
sends a signal to present the first diagnosis and the phenotype keywords associated with the genetic variants on the genome dashboard;
responsive to receiving a signal adding filters from a user of the genome dashboard, applies added filters to the phenotype keywords associated with the genetic variants and the first diagnosis and generates filtered phenotype keywords associated with the genetic variants and generates a second diagnosis; and
sends a signal to present the second diagnosis and the filtered phenotype keywords associated with the genetic variants on the genome dashboard.

2. The genome system of claim 1, wherein the processing device extracts keywords from the multiple text inputs as they are added by the user and extracts additional genetic variants associated with the phenotype keyword as they are created.

3. The genome system of claim 2, responsive to the addition of at least one of extracted keywords and additional genetic variants associated with the phenotype keyword, the processing device generates updated phenotype keywords associated with the genetic variants and a third diagnosis and sends a signal to present the third diagnosis and the updated phenotype keywords associated with the genetic variants on the genome dashboard.

4. The genome system of claim 1, wherein the processing device extracting the phenotype keywords from the multiple text inputs comprises utilizing a natural language processing engine.

5. The genome system of claim 1, wherein the processing device assigns a value to each of the phenotype keywords based upon the genetic variants identified.

6. The genome system of claim 5, wherein the processing device removes phenotype keywords assigned a value below a value threshold and matches the remaining phenotype keywords to the genetic variants present in the genome sequence to generate the first diagnosis.

7. The genome system of claim 1, wherein the processing device consolidates genetic variants having multiple associated phenotypes into a single entry on the interactive genome dashboard.

8. The genome system of claim 7, wherein the processing device removes the single entries lacking an association with at least one of the multiple text inputs and the phenotype keywords to generate one or more final entries.

9. The genome system of claim 8, wherein the processing device creates a vector from vector text for each word of the one or more final entries, sums the vectors to generate a final vector for each word of the one or more final entries, and ranks the one or more final entries based on a cosine distance from the multiple text inputs and the phenotype keywords.

10. The genome system of claim 9, wherein the processing device visually differentiates words of the one or more final entries that resulted in movement of the one or more final entries into a higher rank.

11. A non-transitory computer readable medium storing instructions executable by an associated processor to perform a method for implementing a genome system for displaying an interactive genome dashboard, the method comprising:
storing a first diagnosis generated by the genome system based upon a genome sequence and initial data, the initial data comprising identified genetic variants of the genome sequence, phenotype keywords, multiple text inputs, and phonotype genetic variant associations; and
responsive to receiving additional multiple text inputs:
extracting one or more additional phonotypic terms from the additional multiple text inputs;
identifying one or more genetic variants present in the genome sequence associated with the one or more additional phonotypic terms;
generating a second diagnosis based upon the one or more additional phonotypic terms and the initial data;
responsive to the first diagnosis being the same as the second diagnosis, storing the second diagnosis; and
responsive to the first diagnosis being different than the second diagnosis, presenting the second diagnosis on the genome dashboard.

12. The method of claim 11, wherein responsive to receiving additional phonotype genetic variant associations, the method further comprises:
identifying one or more genetic variants present in the genome sequence associated with the additional phonotype genetic variant associations;
generating a third diagnosis based upon the one or more genetic variants associated with the additional phonotype genetic variant associations and the initial data;
responsive to the first diagnosis being the same as the third diagnosis, storing the third diagnosis; and
responsive to the first diagnosis being different than the second diagnosis, presenting the third diagnosis on the genome dashboard.

13. The method of claim 11, wherein responsive to receiving additional phonotype genetic variant associations, the method further comprises:
identifying phenotype keywords associated with the additional phonotype genetic variant associations;
generating a third diagnosis based upon the phenotype keywords associated with the additional phonotype genetic variant associations and the initial data;
responsive to the first diagnosis being the same as the third diagnosis, storing the second diagnosis; and
responsive to the first diagnosis being different than the third diagnosis, presenting the third diagnosis on the genome dashboard.

14. The method of claim 12, wherein responsive to the generation of at least one of the second diagnosis and the third diagnosis, the method further comprises:
Presenting at least one of the second diagnosis and the third diagnosis on the genome dashboard;
identifying the additional phonotypic terms that formed the basis of the change from the first diagnosis to the at least one of the second diagnosis and the third diagnosis; and
presenting on the genome dashboard the additional phonotype genetic variant associations that were the basis of the change from the first diagnosis to the at least one of the second diagnosis and the third diagnosis.

15. The method of claim 11, wherein responsive to receiving additional phonotype variant associations, the method further comprises:
assigning values to the phenotype variant associations;
removing the additional phenotype variant associations having a value below a value threshold;
altering assigned values based upon received filter selection;
identifying additional genetic variants from the genome that are associated with the additional phenotype variant associations; and
matching the additional genetic variants to phenotype keywords to generate the second diagnosis.

16. The method of claim 12, further comprising applying filters included in the first diagnosis during generation of at least one of the second diagnosis and the third diagnosis.

17. A genome system for displaying an interactive genome dashboard, the genome system comprising:
a processing device having a processor configured to perform a matching function between phenotypes and gene variants to create gene matches based upon one or more text inputs and a genome sequence introduced through the interactive genome dashboard, the processing device:
receives one or more phenotype keywords and the genome sequence of a patient exhibiting the one or more phenotype keywords;
matches and presents on the interactive genome dashboard one or more gene variants present in the genome sequence associated with the one or more phenotype keywords;
identifies and presents on the interactive genome dashboard disease candidates based upon the one or more gene variants association with the one or more phenotype keywords;
identifies and presents on the interactive genome dashboard non-represented gene variants that are associated with each of the disease candidates that are not present in the one or more gene variants; and
generates a sortable list on the interactive genome dashboard for identifying each of the one or more phenotype keywords and each of the one or more gene variants that comprises clinical evidence supporting each of the disease candidates.

18. The genome system of claim 17, the processing device identifying non-diagnosing gene variants from the one or more gene variants, the non-diagnosing gene variants neither confirm or deny a diagnosis of the disease candidates.

19. The genome system of claim 18, responsive to the processing device identifying non-diagnosing gene variants from the one or more gene variants, the processing device presents the non-diagnosing gene variants on the sortable list, wherein the non-diagnosing gene variants are presented with at least one of an annotation and a visual indicator.

20. The genome system of claim 18, responsive to receiving additional one or more gene variants associated with the one or more phenotype keywords, the processing device:
matches and presents on the interactive genome dashboard additional one or more gene variants present in the genome sequence associated with the one or more phenotype keywords;
identifies additional disease candidates based upon the additional one or more gene variants associated with the one or more phenotype keywords;
responsive to the disease candidates being the same as the additional disease candidates, storing the additional disease candidates; and
responsive to the disease candidates being different than the additional disease candidates, presenting the additional disease candidates on the genome dashboard.
